(19) United States
(12) Patent Application Publication
(10) Pub. No.: US 2001/0032648 A1
(43) Pub. Date: Oct. 25, 2001

(54) Exhaust airway for nasal continuous positive airway pressure mask

(76) Inventor: Bernadette Jestrabek-Hart, Meridian, ID (US)

Correspondence Address: PEDERSEN & COMPANY, PLLC, P.O. BOX 2666, BOISE, ID, 83701, US

(21) Appl. No.: 09/800,602

(22) Filed: Mar. 5, 2001

Related U.S. Application Data
(60) Provisional application No. 60/186,857, filed on Mar. 3, 2000.

(51) Int. Cl. A62B 7/00; A61M 16/00; F16K 31/02; A62B 18/02

(52) U.S. Cl. 128/204.23; 128/205.25

(57) ABSTRACT

The present invention is an exhaust airway for a continuous positive airway pressure mask for assisting in the treatment of sleep apnea. The present invention generally comprises an improved structure and method of exhausting air from the nosepiece of the mask, which structure and method can be incorporated into most standard CPAP respiratory masks, to keep the exhaust air away form the user and/or the user's bed partner. An elongated exhaust passage is provided, separate from the inlet air passage but preferably attached or molded into the respiratory mask intake hose. The exhaust airway has an intake hole on the respiratory mask at or near where the intake air hose attaches to the mask. The exhaust airway runs along the intake air hose for about 7 inches or more and then ends with an exit hole from which the exhaust air actually escapes. The invention provides an exhaust airway separate from the inlet airway, to preferably keep the exhaust air completely away from the face and eyes and away from bouncing off the bedding and back onto the wearer or the wearer's bed partner. It also allows the wearer to lay with his face into the pillow while sleeping and still allow the respiratory mask to work efficiently.

This application claims priority of my prior provisional application, Serial 60/186,857, filed Mar. 3, 2000, which is hereby incorporated herein by reference.

### BACKGROUND OF THE INVENTION

1. Field of the Invention

The present invention generally relates to medical equipment. More specifically, the present invention relates to the exhaust air that comes out of the masks used in the treatment of sleep apnea.

2. Related Art

Sleep apnea is a breathing disorder characterized by brief interruptions of breathing during sleep. Certain mechanical and structural problems in the airway of a person cause the interruptions in breathing during sleep. In some people, apnea occurs when the throat muscles and tongue relax during sleep and partially block the opening of the airway. When the muscles of the soft palate at the base of the tongue and the uvula relax and sag, the airway becomes blocked, making breathing labored and noisy and even stopping it altogether. Sleep apnea also can occur in obese people when an excess amount of tissue in the airway causes the airway to be narrowed. With a narrowed airway, the person continues his or her efforts to breathe, but air cannot easily flow into or out of the nose or mouth. Unknown to the person, this results in heavy snoring, periods of no breathing, and frequent arousals, which are abrupt changes from deep sleep to light sleep. See Facts About Sleep Apnea, National Institute of Health, Publication No. 95-3798, September 1995.

During the apneic event, the person is unable to breathe in oxygen and to exhale carbon dioxide, resulting in low levels of oxygen and increased levels of carbon dioxide in the blood. The reduction in oxygen and increase in carbon dioxide alert the brain to resume breathing, and cause an arousal. With each arousal, a signal is sent from the brain to the upper airway muscles to open the airway breathing is resumed, often with a loud snort or gasp. Frequent arousals, although necessary for breathing to restart, prevent the patient from getting enough restorative deep sleep.

Nasal continuous positive airway pressure (CPAP) is the most common effective treatment of sleep apnea. In this procedure, the patient wears a mask over the nose during sleep, and pressure from an air blower forces air through the nasal passages. The air pressure is adjusted so that it is just enough to prevent the throat from collapsing during sleep. The pressure is constant and continuous. Nasal CPAP prevents airway closure while in use, but apnea episodes return when CPAP is stopped or used improperly. Variations of the CPAP device attempt to minimize side effects that sometimes occur, such as nasal irritation and drying, facial skin irritation, abdominal bloating, mask leaks, sore eyes, and headaches.

CPAP devices are illustrated in the patent literature. Landis (U.S. Pat. No. 5,687,715) and Handke, et al. (U.S. Pat. No. 5,724,965) illustrate two approaches to CPAP. In Handke, et al., one may see the conventional vents, which are short, outwardly-protruding nozzles on the mask nosepiece directly on each side of the inlet hose connection to the nosepiece. With such short vents directing exhaust air straight out from the face at either side of the nose, the exhaust air tends to flow against a pillow and turbulently flow back into/against the eyes and cheeks. Handke, et al., especially, illustrates how conventional CPAP masks work and may be constructed. Alternative conventional exhaust vents may be a grouping of holes or diffusing holes clustered on the surface of the nosepiece within about 1 inch below the inlet hose or closely around the inlet hose also within about 1 inch of the inlet hose.

Still, there is needed an improved exhaust system for application to conventional CPAP masks. There is a need to supply a convenient and comfortable CPAP mask that does not direct a direct or deflected stream of exhaust air against the user's or a sleeping partner's face or eyes.

### SUMMARY OF THE INVENTION

The present invention is a improvement in the handling of exhaust air in a CPAP mask used for assisting in the treatment of sleep apnea. The present invention may be applied to various standard CPAP masks that are supplied with air under pressure by an air blower or other source. The preferred mask is the type with a single inlet hose entering a nosepiece, wherein the nosepiece receives the nose in a central cavity and seals around the nose against the cheeks and upper lip.

The present invention generally comprises an exhaust airway providing an improved way of directing the exhaust air away from the user's face and eyes, and away from hitting any bedding or other surface near the face and then blowing back into the user's face or body. The exhaust airway also helps prevent the exhaust air from blowing onto a sleeping partner of the CPAP wearer.

The main problems with prior CPAP exhaust systems result from the fact that the exhaust from the mask blows out proximally to the user's face, within about an inch or less of the outer surface of the mask nosepiece. Exhaust air typically flows out from short vents/apertures on the surface of the nosepiece of the CPAP mask. Thus, the exhaust air flows out close to and directly in front of the face, near the middle of the face in the nose or cheek area. If the user lies in any position other than on his/her back, then the air hits the bedding or part of the wearer and deflects back onto the user, or the air hits a partner that sleeps with the wearer. The worst results from this air are the feeling that a jet of air is intermittently blowing on your skin and the dry eye that can result.

The invented exhaust airway comprises a tube for conducting exhaust air from the cavity of a CPAP mask away from the cheek and nose area to a distance where the exhaust air will dissipate without drying, chaffing, or irritating the user. Preferably, the invented exhaust airway comprises a tube that is separate from the inlet air conduit that delivers air to the mask. The exhaust tube is in fluid communication with the cavity of the mask, and hence, in fluid communication with the nose. The exhaust tube conducts the exhaust air away from the mask, and preferably down to about the mid-neck or upper shoulder area, or up to the top of the forehead or to the top-of-the-head area.

In use, the air supply provides a positive pressure of fresh air to the mask cavity for breathing in by the user. When the user exhales into the cavity, the cavity “vents” by exhaust air flowing out of the cavity into the exhaust tube, so that the exhaust gas coming out of the exhaust tube is typically a mixture of the gases being exhaled and air continuously being supplied to the cavity. The exhaust tube is sized to achieve the net effect of “fresh” air being supplied to the user at all times, but that the masks efficiently and comfortably exhausts the exhaled gases.

### BRIEF DESCRIPTION OF THE DRAWINGS

FIG. 1 is a right side perspective view of one embodiment of a CPAP mask on a user, with one embodiment of the invented exhaust airway incorporated into the mask.

FIG. 2 is a left side perspective of another embodiment of a CPAP mask on a user, with an embodiment of the invented exhaust airway incorporated into the mask.

FIG. 3 is a left side view of another embodiment of a CPAP mask on a user, including an alternative embodiment of the invented exhaust airway.

FIG. 4 is a right side view of another embodiment of a CPAP mask on a user, including an embodiment of the invented exhaust airway that is similar to the airway of FIG. 3.

FIG. 5a is a schematic, radial cross-sectional view of one embodiment of the invented combination of an intake hose (D) and invented exhaust tube (C), which combination is formed as part of the respiratory mask molding process.

FIGS. 5b is a schematic, side view of the combination intake hose and exhaust tube of FIG. 5a.

FIGS. 6a and 6b are radial cross-sectional views of other embodiments of the invented combination of an intake hose (D) and exhaust tube (C), wherein the exhaust tubes are made or molded separately from the intake hose and then secured to the intake air hose.

FIG. 7a is a schematic, cross-sectional view from the side of one embodiment of the invented mask with exhaust airway, shown during inhaling by the user.

FIG. 7b is a schematic, cross-sectional view from the side of one embodiment of the invented mask with exhaust airway, shown during exhaling by the user.

### DESCRIPTION OF THE PREFERRED EMBODIMENTS OF THE INVENTION

Referring to the Figures, there are shown several, but not the only, embodiments of the invented exhaust airway for a Continuous Positive Airway Pressure (CPAP) mask. The exhaust airway comprises a elongated tube in fluid communication with the interior cavity 32 of the mask nosepiece 34 in which the user places his/her nose to breath the supply of air delivered to the interior cavity under positive pressure by the blower or other source 35. The elongated tube 30 extends away from the nosepiece 34 a distance and a direction that helps prevent the exhaust air moving through and out of it from blowing on the user or on the surrounding pillow or bedding and then back onto the user. The preferred distance for the outlet of the exhaust tube from the mask nosepiece is about 7 inches or more, and preferably about 7-12 inches. The preferred direction for the exhaust tube 30 to extend is generally parallel to the face downward to about neck level or upward to about forehead level. The outlet of the exhaust tube is preferably pointed straight outward perpendicular to the face a significant distance from the nosepiece, preferably 7 or more inches.

The exhaust tube 30 is a conduit separate from the inlet conduit (inlet hose 26) but is preferably attached to the inlet hose or integrally molded with the inlet hose with a partition wall 37 between the inlet air passage 20 and the exhaust passage 22. Preferably, the exhaust tube extends parallel and close to the intake hose, all the way along the length of the exhaust tube.

Intake hoses may be attached to the nosepiece by various means, and the exhaust tube may be attached to the nosepiece by similar means, for example, integral molding, attachment by way of the seal against an aperture in the nosepiece, a swivel connection, etc. Preferably, no leakage of air comes out from the nosepiece by any other path except the exhaust tube. Preferably, therefore, in a retrofit situation, the conventional vents of the mask become the attachment point and are covered by the exhaust tube, so that the conventional vents become the inlet(s) for the exhaust tube. The connection of the exhaust tube to the conventional vents (or to another orifice through the nosepiece in the OEM integral embodiments) is made air-tight, so that all or substantially all of the exhaust air travels through the exhaust tube and is delivered a substantial distance from the user's cheeks and eyes.

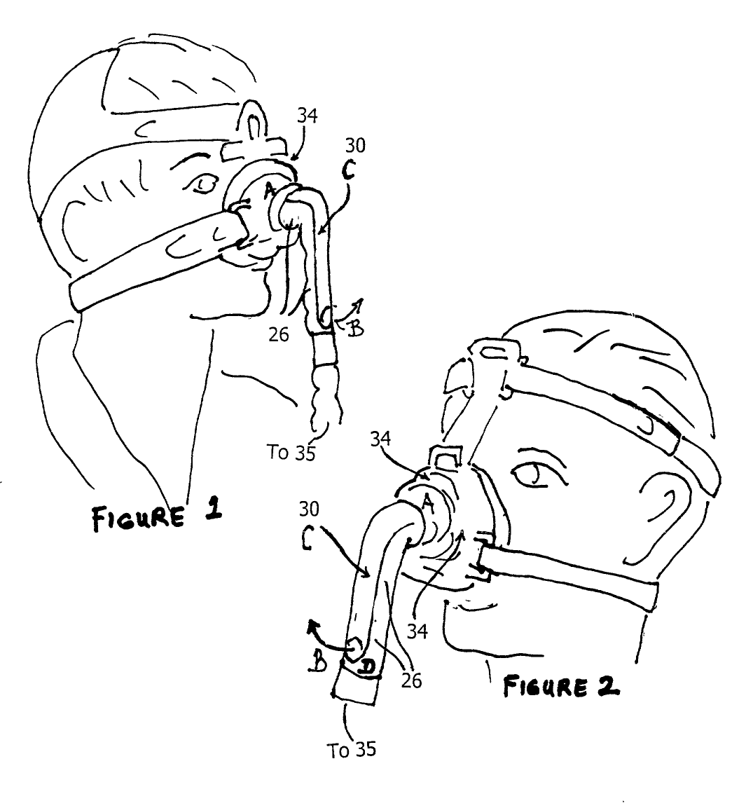

As shown in the Figures, the present invention is an addition to currently available respiratory masks used for assisting in the treatment of sleep apnea. The preferred embodiment of the present invention, shown in FIGS. 1, 2, 3, (made as part of the respiratory mask mold instead of a completely separate, attached tube), & FIG. 4 (example of a completely separate, attached tube), comprises a separate airway tube (C) that is created as part of or lies along the intake hose (D) having an opening placed inside the respiratory mask (at A) into which the exhaust air enters and then travel along this additional, separate airway tube to an exit opening (B) that lies at least 7 inches or more away from where the entrance exhaust airway starts. The size of this exhaust airway passage will be determined by the amount of air that has to escape to make the respiratory mask complete its function of keeping pressure enough to prevent the user's throat from collapsing during sleep.

FIG. 1 illustrates an embodiment of the elongated exhaust tube (C) designed to cooperate with a swivel intake hose (D). This embodiment of exhaust tube (C) is built into the intake hose preferably at the swivel (close to connection position A), so that the tube (C) can swivel along with the hose (D). The exhaust tube curves out along the intake hose, as the intake hose turns about 90 degrees downward. At about mid-neck level or lower, exhaust tube (C) has exit hole (B), about 7 inches or more from the connection of the tube (C) to the actual mask (the nosepiece). One may see in FIG. 1 that the exhaust tube may generally align with the center of the user's chin, but, due to the swivel, the tube may also swivel about 360 degrees around the axis of rotation at the hose/tube connection to the nosepiece. The diameter of an exhaust tube (C) of a desired length, in this embodiment and others, is determined by the amount of air that has to escape to allow the respiratory mask to complete its functions of maintaining air pressure enough in the interior cavity of the nosepiece to prevent the user's throat from collapsing during sleep, and its function of venting exhales gases adequately to maintain an air composition in the interior cavity in an appropriate range of oxygen levels.

FIG. 2 illustrates another mask designed with a swivel intake hose (D) showing the exhaust tube (C) built into the intake hose from a swivel (connection position A), and extending out along the intake hose with an exit hole (B) at 7 inches or more from the actual mask. As in FIG. 1, this exhaust airway follows generally along with the intake hose, preferably by being molding into a single unit with the inlet hose with a partition wall 37 between the longitudinal passage 20 of the hose and the longitudinal passage 22 of the tube.

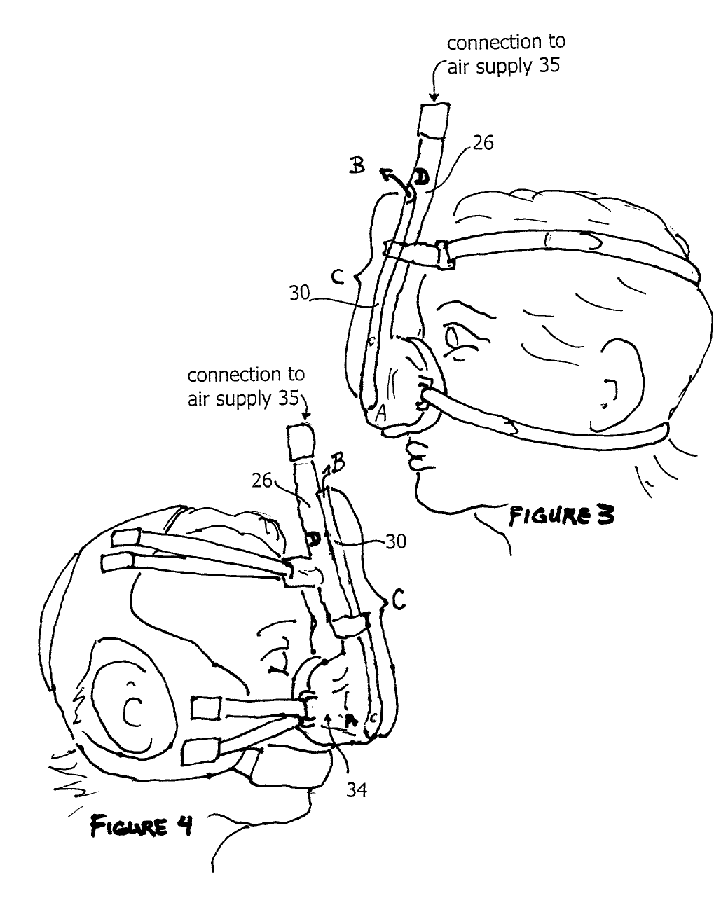

FIG. 3 illustrates another style of mask, which is similar to the Mirage™ mask by Respironics, wherein the invented exhaust tube (C) is included OEM to be integral with the inlet hose, the mask starting at the base of the nose on the respiratory mask (connection position A) and extending along the mask and the intake hose (D) with a exit hole (B) seven or more inches from the actual respiratory mask. In such a design, the exhaust tube may be integral with the mask and inlet hose, with a partition wall 37 separating the inlet hose and the exhaust tube to the keep the exhaust air separate from the inlet air. In this embodiment and other embodiments, the outlet of the inlet hose may be vary close to the inlet of the exhaust tube, as long as the flows of inlet air and exhaust are balanced properly by the supply of air and the relative diameters/cross-sectional areas of the hose and tube. In the case of the swivel-style respiratory mask, shown in FIG. 1, 2, & 3, the exit airway tube (C) should be run along the top and outer surface of the inlet hose.

FIG. 4 illustrates another style of mask similar to the Mirage™ mask, which, before additional of a retrofit exhaust tube according to the invention, has a plurality of vent holes through the nosepiece below the “nose base” of the nosepiece. In FIG. 4, an exhaust tube according to the invention has been added to the Mirage™ mask, showing the exhaust vent tubing (C) added onto the outside of the mask covering and fluidly communication with the plurality of vent holes. The exhaust tube, therefore, starts at the base of the nose on the respiratory mask (at connection position A), and extends along the outside of the mask and the intake hose (D) with a exit hole (B) seven or more inches from the actual respiratory mask. This is a “retrofit” or “non-integral” style that may be made by using a separate mold for the exhaust tube, for example, and then by attaching the tube to the mask and inlet hose.

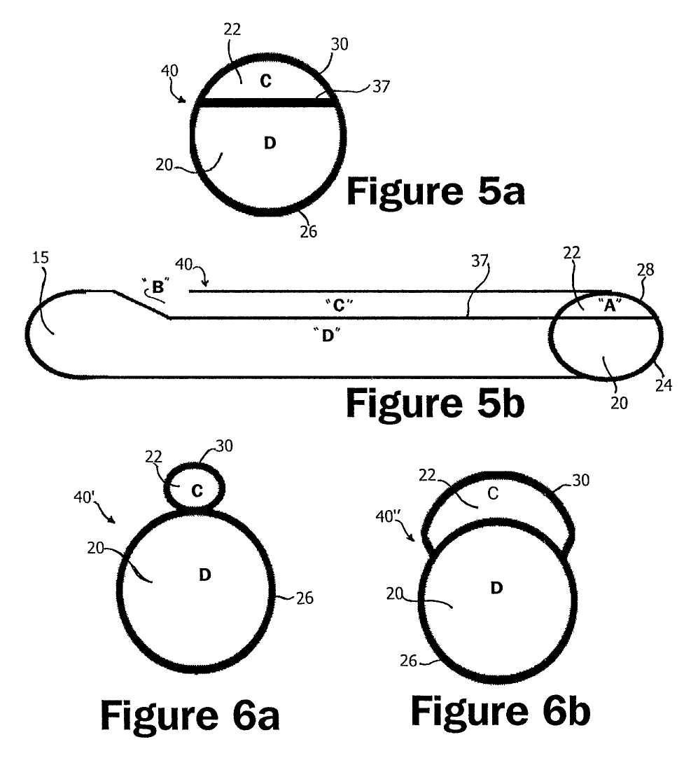

FIGS. 5a, 5b, 6A, and 6b schematically illustrate the relationship of various embodiments of the exhaust tube to the inlet hose. In FIG. 5a, the inlet hose/exhaust tube combination 40 is a single hollow cylindrical tube, with a single partition wall 37 running a length along the hose to divide the passage of the exhaust tube (C) from the passage of the inlet air hose (D). The configuration of FIG. 5a may be further illustrated as in FIG. 5b, which shows a supply end 15 of the inlet hose that connects to the air supply, the hose outlet end 24 that connects to the nosepiece 34, the exhaust tube inlet end 28 that connects to the nosepiece and the exhaust tube outlet/exit hole (B). The exhaust passage outlet/exit hole (B) is along the side of the hose/tube combination 40, several inches preferably 7 or more) along the inlet hose from the end (24,28) that is connected to the nosepiece. Alternatively, but less preferably, the exhaust tube may be inside and concentric with the inlet hose.

In FIGS. 6a and 6b are shown cross-sectional view of alternative combinations of hose and tube. These hose/tube combinations 40′, 40″ are of the retrofit, add-on style, wherein the exhaust tube appears to be added onto the outside of the cylindrical inlet hose. The exhaust tube may be of various shapes, for example, tubular/cylindrical in FIG. 6a, or semi-cylindrical as in FIG. 6b. Others may also be used. The exhaust tube is typically significantly smaller in diameter or smaller in cross-sectional area, than the inlet hose, to control the smaller flowrates through the tube.

In “add-on” embodiments, in which the exhaust tube is not built integrally into the inlet hose, the exhaust tube may be attached securely to the outside of the existing intake airway hose with the exhaust tube inlet end firmly connected to the mask exhaust hole(s), at or near the nosepiece outer surface, so the exhaust air does not escape at or near the mask, rather the exhaust air escapes on the other, exit end of the attached hose, therefor making this attached airway the actual exhaust part of the respiratory mask.

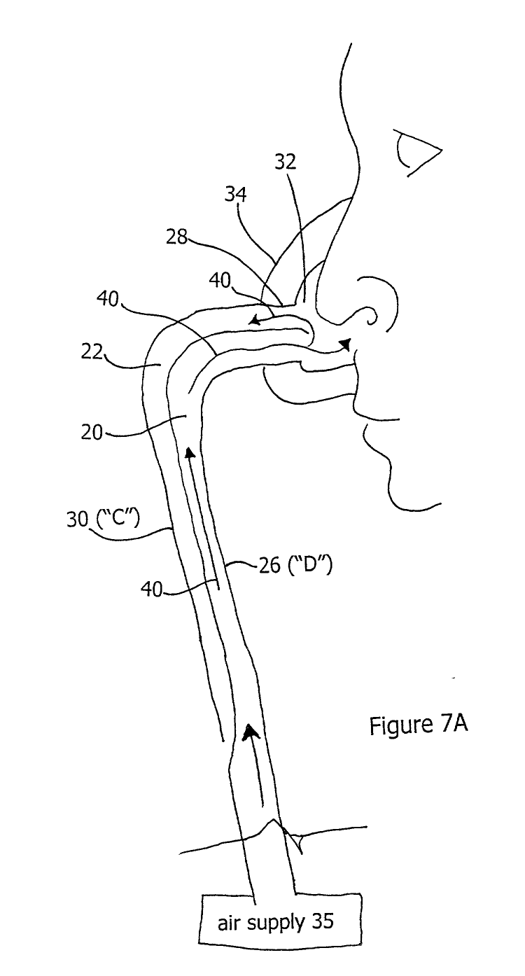

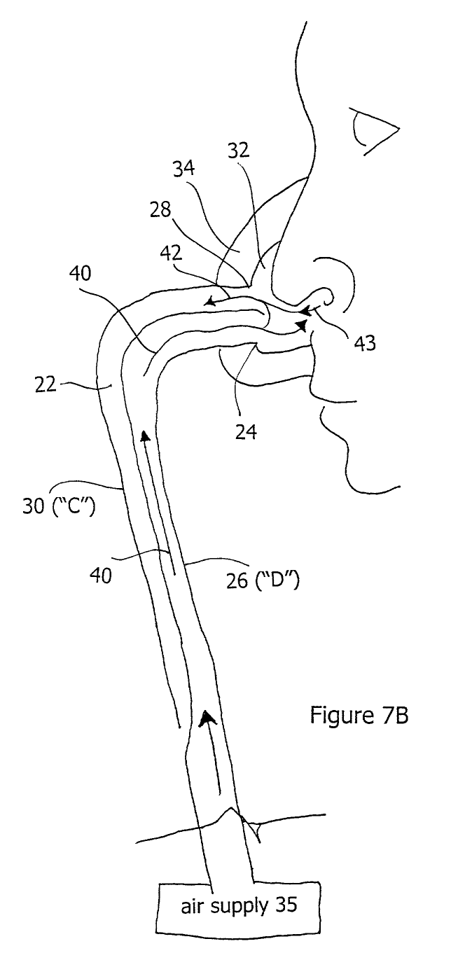

FIGS. 7a and 7b schematically illustrate the structure of one embodiment of the mask with exhaust airway, during inhaling and exhaling, respectively. One may see that the inlet air passageway 20 is separate from the exhaust passageway 22 preferably all the way along the length of the exhaust tube. The outlet end 24 of the inlet hose 26 and the inlet end 28 of the exhaust tube 30 both connect and fluidly communicate with the cavity 32 of the nosepiece 34. The flows of air 40 and air mixture 42 (including exhaled gas 43) are determined by such factors as air supply pressure, by hose and tube cross-sectional area, and by any optional valving or on-way valving (not shown) provided.

The Figures illustrate applications of the invented exhaust airway, and details of said airway. The exhaust tube may be a passage through and parallel with the inlet hose, not necessarily noticeably visible from the outside of the hose except for the exhaust tube outlet hole. The exhaust tube may be a separate tube distinctly visible as a parallel tube on the outside of the inlet hose. Regardless the style of the mask, the exhaust tube with its exit hole preferably is positioned somewhere along the intake hose, at least 7 inches or more, away from the mask. The guideline of 7 inches or more results in most people enjoying the comfortable effects of the invention. This will keep the exhaust air from hitting the wearer's face, arms, partner or bedding and bouncing off, back onto the wearer. It will also allow the wearer to sleep in other positions without restricting the exhaust exit flow.

By “air supply” in this description and the claims, the inventor does not intend to limit the invention to being used only with room or ambient air supplies, but also, for example, other gases, such as air plus an amount of added oxygen.

Although this invention has been described above with reference to particular means, materials and embodiments, it is to be understood that the invention is not limited to these disclosed particulars, but extends instead to all equivalents within the scope of the following claims.

I claim:

1. A respiratory mask for treating sleep apnea, the respiratory mask comprising:
a nosepiece having a cavity for placement over a user's nose;
an inlet hose having a length, an outlet end, and a supply end, and an inlet air passageway, the outlet end attached to the nosepiece and fluidly communicating with the cavity, and the supply end adapted to communicate with an air supply, so that the inlet hose delivers a positive pressure of air into the cavity for the user to breath;
an exhaust tube having a length, an inlet end, and an exhaust end, and an exhaust air passageway, the inlet end attached to the nosepiece and fluidly communicating with the cavity, the exhaust tube length being about 7 inches or greater so that the exhaust tube extends away from the nosepiece to hold the exhaust end about 7 inches or more away from the nosepiece;
wherein the exhaust tube conducts exhaust air away from the user's face for increasing comfort during sleep.

2. The respiratory mask as in claim 1, wherein the exhaust tube is connected to the inlet hose substantially all along the length of the exhaust tube.

3. The respiratory mask as in claim 1, wherein the exhaust tube and the inlet hose share a common sidewall as a partition between the inlet air passageway and the exhaust air passageway.

4. The respiratory mask as in claim 1, wherein the inlet hose and the exhaust tube extend upward from the nosepiece to an area near the user's forehead.

5. The respiratory mask as in claim 1, wherein the inlet hose and the exhaust tube extend downward from the nosepiece to an area near the user's neck.

6. The respiratory mask as in claim 1, wherein the inlet hose and the exhaust tube are integrally molded into a single unit for attachment to the nosepiece.

7. A respiratory mask for treating sleep apnea, the respiratory mask comprising:
a nosepiece having a cavity for placement over a user's nose;
an inlet hose having a length, an outlet end, and a supply end, and an inlet air passageway, the outlet end attached to the nosepiece and fluidly communicating with the cavity, and the supply end adapted to communicate with an air supply, so that the inlet hose delivers a positive pressure of air into the cavity for the user to breath; and
an exhaust tube having a length, an inlet end, and an exhaust end, and an exhaust air passageway, the inlet end attached to the nosepiece and fluidly communicating with the cavity, and the exhaust tube extending generally parallel to the inlet hose;
wherein the inlet air passageway is separate from the exhaust air passageway, so that an air mixture in the cavity is vented through the exhaust tube; and
wherein the exhaust tube conducts exhaust air away from the user's face for increasing comfort during sleep.

8. The respiratory mask as in claim 7, wherein the exhaust tube is connected to the inlet hose substantially all along the length of the exhaust tube.

9. The respiratory mask as in claim 7, wherein the exhaust tube and the inlet hose share a common sidewall as a partition between the inlet air passageway and the exhaust air passageway.

10. The respiratory mask as in claim 7, wherein the inlet hose and the exhaust tube extend upward from the nosepiece to an area near the user's forehead.

11. The respiratory mask as in claim 7, wherein the inlet hose and the exhaust tube extend downward from the nosepiece to an area near the user's neck.

12. The respiratory mask as in claim 7, wherein the inlet hose and the exhaust tube are integrally molded into a single unit for attachment to the nosepiece.

13. A method of increasing sleeping comfort during use of a respiratory mask used for treating sleep apnea, the method comprising:
providing air to a nosepiece surrounding a user's nose through an inlet hose that extends outwardly from the nosepiece and depends downwardly from the nosepiece; and
exhausting an air mixture comprising exhaled air from the nosepiece through an exhaust tube that runs generally parallel to the inlet tube outward from the nosepiece and depending down from the nosepiece a distance from the nosepiece to vents the air mixture to the atmosphere below the nosepiece at least 7 inches from the nosepiece.

14. The method of claim 14, wherein the exhaust tube has an outlet end distanced from the nosepiece, and the method further comprises venting the air mixture straight outward from the user at least 7 inches below the nosepiece.

15. A method of increasing sleeping comfort during use of a respiratory mask used for treating sleep apnea, the method comprising:
providing air to a nosepiece surrounding a user's nose through an inlet hose that extends outwardly from the nosepiece and upends upwardly from the nosepiece; and
exhausting an air mixture comprising exhaled air from the nosepiece through an exhaust tube that runs generally parallel to the inlet tube outward from the nosepiece and upending up from the nosepiece a distance from the nosepiece to vents the air mixture to the atmosphere above the nosepiece at least 7 inches from the nosepiece.

16. The method of claim 15, wherein the exhaust tube has an outlet end distanced from the nosepiece, and the method further comprises venting the air mixture straight outward from the user at least 7 inches above the nosepiece.
